(19) United States
(12) Patent Application Publication
(10) Pub. No.: US 2020/0270547 A1
(43) Pub. Date: Aug. 27, 2020

(54) MICROBIAL CULTURE, COMPOSITION, USE AND METHOD

(71) Applicant: NOVOZYMES A/S, Bagsvaerd (DK)

(72) Inventors: Rasmus Rune Hansen, Ballerup (DK); Jennifer Nelson, Roanoke, VA (US); Thomas Amos, Roanoke, VA (US)

(73) Assignee: NOVOZYMES A/S, Bagsvaerd (DK)

(21) Appl. No.: 16/075,010

(22) PCT Filed: Feb. 7, 2017
(86) PCT No.: PCT/US2017/016814

§ 371 Date: Aug. 2, 2018

Related U.S. Application Data
(60) Provisional application No. 62/295,250, filed on Feb. 15, 2016.

(51) Int. Cl. C11D 3/38; C12N 1/20; C12R 1/125; C12R 1/01; C12R 1/38; C11D 3/00; C11D 3/386

(52) U.S. Cl. CPC C11D 3/381; C12N 1/20; C12R 1/125; C11D 3/38627; C12R 1/38; C11D 3/0015; C12R 1/01

(57) ABSTRACT

The present invention concerns one or more bacteria cultures of the genus Bacillus, Lysinibacillus or Pseudomonas, a composition comprising the bacteria cultures, the use of the bacteria cultures for preventing and/or reducing amount of free fatty acids and a method for washing.

### REFERENCE TO A DEPOSIT OF BIOLOGICAL MATERIAL

This application contains a reference to a deposit of biological material, which deposit is incorporated herein by reference.

### FIELD OF THE INVENTION

The present invention concerns one or more isolated bacteria cultures of the genus Bacillus, Lysinibacillus or Pseudomonas, a composition comprising the bacteria cultures, the use of the bacteria cultures for preventing and/or reducing amount of free fatty acids and a method for washing.

### BACKGROUND OF INVENTION

Consumers prefer that items, such as garments, be as clean as possible. Such consumers typically associate the odor of a cleaned or treated article with the degree of cleanliness of such item. Thus, the effectiveness of a cleaning and/or treatment composition, from a consumer's perspective, is typically directly linked with the odor that such composition imparts to an item that is cleaned or treated with such composition.

Esterases and lipases are particularly effective cleaning agents and are used widely in detergent composition for laundry or hard surface cleaning. These enzymes can generate objectionable fatty acid odors because they degrade lipid present on the item to free fatty acids. In particular short-chain fatty acids results in undesired odors such as the odor of butyric acid and caproic acid. The consumers therefore often associate these odors with lack of cleanliness. The odor is primarily developed after the wash process, in the drying phase where the activity of the lipase increases due to the decreasing water content.

### SUMMARY OF THE INVENTION

The present invention concerns an isolated culture having characteristics substantially identical to that of a strain selected from the group consisting of:
the strain having the deposit accession number NRRL-B-67164;
the strain having the deposit accession number NRRL B-50256;
the strain having the deposit accession number NRRL-B-67163;
the strain having the deposit accession number NRRL-B-67162;
the strain having the deposit accession number NRRL-B-67160;
the strain having the deposit accession number NRRL-B-67161;
a mutant of at least one of the deposited strains, wherein the mutant has all of the identifying characteristics of the corresponding deposited strain;
a progeny of one of the deposited strains; or
a mixture of two or more of the strains, mutants, or progeny.

The invention further concerns a composition comprising one or more isolated cultures species selected from the group consisting of Bacillus subtilis, Bacillus subtilis subsp. subtilis, Lysinibacillus contaminans or Pseudomonas monteilii.

Further, the invention concerns the use of one or more isolated bacteria cultures for reducing the amount of free fatty acids present on an item, wherein the bacteria culture is a strain of the genus Bacillus, Lysinibacillus or Pseudomonas.

The invention also concerns a method for washing an item comprising the steps of:

a. Exposing the item to a wash liquor;

b. Completing at least one wash cycle; and

c. Optionally rinsing the item with water optionally comprising a rinse aid,

wherein the wash liquor and/or the rinse aid comprise one or more isolated cultures of the invention or a composition of the invention.

### Definitions

Detergent Component: The detergent component is different to the bacteria cultures of this invention. The precise nature of these detergent components, and levels of incorporation thereof, will depend on the physical form of the composition and the nature of the operation for which it is to be used. Suitable detergent components include, but are not limited to the components described below such as surfactants, builders, flocculating aid, chelating agents, dye transfer inhibitors, enzymes, enzyme stabilizers, enzyme inhibitors, catalytic materials, bleach activators, hydrogen peroxide, sources of hydrogen peroxide, preformed peracids, polymeric dispersing agents, clay soil removal/anti-redeposition agents, brighteners, suds suppressors, dyes, perfumes, structure elasticizing agents, fabric softeners, carriers, hydrotropes, builders and co-builders, fabric huing agents, anti-foaming agents, dispersants, processing aids, and/or pigments.

Detergent Composition: The term “detergent composition” refers to compositions that find use in the removal of undesired compounds from items to be cleaned, such as textiles or hard surfaces. The detergent composition may be used to, e.g., clean textiles for both household cleaning and industrial cleaning. The terms encompass any materials/compounds selected for the particular type of cleaning composition desired and the form of the product (e.g., liquid, gel, powder, granulate, paste, or spray compositions) and includes, but is not limited to, detergent compositions (e.g., liquid and/or solid laundry detergents and fine fabric detergents; fabric fresheners; fabric softeners; and textile and laundry pre-spotters/pretreatment). In addition to containing the bacteria culture of the invention, the detergent composition may contain one or more additional enzymes (such as amylases, catalases, cellulases (e.g., endoglucanases), cutinases, DNases, haloperoxygenases, lipases, mannanases, pectinases, pectin lyases, peroxidases, proteases, xanthanases, xyloglucanases, or any mixture thereof), and/or detergent adjunct ingredients such as surfactants, builders, chelators or chelating agents, bleach system or bleach components, polymers, fabric conditioners, foam boosters, suds suppressors, dyes, perfume, tannish inhibitors, optical brighteners, bactericides, fungicides, soil suspending agents, anti-corrosion agents, enzyme inhibitors or stabilizers, enzyme activators, transferase(s), hydrolytic enzymes, oxido reductases, bluing agents and fluorescent dyes, antioxidants, and solubilizers.

Dishware: The term “dishware” is intended to mean any form of kitchen utensil, dinner set or tableware such as but not limited to pans, plates, cups, knives, forks, spoons, porcelain etc. The dishware can be made of any suitable material such as metal, glass, rubber, plastic, PVC, acrylics, ceramics, china or porcelain.

Hard surface: The term “hard surface” is defined herein as hard surfaces including floors, tables, walls, roofs etc. as well as surfaces of hard objects such as cars (car wash) and dishes (dishware). The term “hard surface” includes also the surfaces in the interior of washing machines, such as the interior of laundry washing machines or dishwashing machines, this includes soap intake box, walls, windows, baskets, racks, nozzles, pumps, sump, filters, pipelines, tubes, joints, seals, gaskets, fittings, impellers, drums, drains, traps, coin traps inlet and outlets. The term hard surface does not encompass textile or fabric.

Isolated: The term “isolated” means a substance in a form or environment that does not occur in nature. Non-limiting examples of isolated substances include (1) any non-naturally occurring substance, (2) any substance including, but not limited to, any microorganism, bacteria or fungi, that is at least partially removed from one or more or all of the naturally occurring constituents with which it is associated in nature; or, (3) any substance modified by the hand of man relative to that substance found in nature. An isolated substance may be present in a fermentation broth sample or in a suitable carrier substance.

Laundering: The term “laundering” relates to both household laundering and industrial laundering and means the process of treating textiles with a solution containing a cleaning or detergent composition of the present invention. The laundering process can for example be carried out using, e.g., a household or an industrial washing machine or can be carried out by hand.

Rinse aid: The term “rinse aid” means a composition that can be used together with water during a rinse cycle after washing an item. Examples of rinse aids are fabric softeners/fabric conditioners that are used in the rinse water when a laundry item is rinsed after being washed. Another example is dishware washed in an automated dishwashing machine, where the dishware is rinsed with water comprising a rinse aid. The rinse aid is different from the bacteria culture of the invention.

Textile: The term “textile” means any textile material including yarns, yarn intermediates, fibers, non-woven materials, natural materials, synthetic materials, and any other textile material, fabrics made of these materials and products made from fabrics (e.g., garments and other articles). The textile or fabric may be in the form of knits, wovens, denims, non-wovens, felts, yarns, and toweling. The textile may be cellulose based such as natural cellulosics, including cotton, flax/linen, jute, ramie, sisal or coir or manmade cellulosics (e.g., originating from wood pulp) including viscose/rayon, cellulose acetate fibers (tricell), lyocell or blends thereof. The textile or fabric may also be non-cellulose based such as natural polyamides including wool, camel, cashmere, mohair, rabbit and silk or synthetic polymers such as nylon, aramid, polyester, acrylic, polypropylene and spandex/elastane, or blends thereof as well as blends of cellulose based and non-cellulose based fibers. Examples of blends are blends of cotton and/or rayon/viscose with one or more companion material such as wool, synthetic fiber (e.g., polyamide fiber, acrylic fiber, polyester fiber, polyvinyl chloride fiber, polyurethane fiber, polyurea fiber, aramid fiber), and/or cellulose-containing fiber (e.g., rayon/viscose, ramie, flax/linen, jute, cellulose acetate fiber, lyocell). Fabric may be conventional washable laundry, for example stained household laundry. When the term fabric or garment is used, it is intended to include the broader term textiles as well. In the context of the present invention, the term “textile” also covers fabrics.

Wash cycle: The term “wash cycle” is defined herein as a washing operation, wherein an item such as a textile/fabric or hard surface is exposed to the wash liquor, mechanical action of some kind is applied to the textile/fabric/hard surface in order to release stains and to facilitate flow of wash liquor in and out or on the item and finally the superfluous wash liquor is removed. After one or more wash cycles, the item is generally rinsed and dried.

Wash liquor: The term “wash liquor” is defined herein as a solution of detergent components in water or a mixture of water and detergent components optionally including the bacteria culture of the invention.

### DETAILED DESCRIPTION OF THE INVENTION

The present invention concerns an isolated culture having characteristics substantially identical to that of a strain selected from the group consisting of:
the strain having the deposit accession number NRRL-B-67164;
the strain having the deposit accession number NRRL B-50256;
the strain having the deposit accession number NRRL-B-67163;
the strain having the deposit accession number NRRL-B-67162;
the strain having the deposit accession number NRRL-B-67160;
the strain having the deposit accession number NRRL-B-67161;
a mutant of at least one of the deposited strains, wherein the mutant has all of the identifying characteristics of the corresponding deposited strain;
a progeny of one of the deposited strains; or
a mixture of two or more of the strains, mutants, or progeny.

The inventors have surprisingly found that these bacterial strains are beneficial in laundry processes, where a lipase is used for the removal of lipid stains from laundry items. The inventors have found that the strains are capable of reducing the amount of free fatty acids from laundry items comprising one or more free fatty acids, such as laundry items with stains comprising lipid that are washed with a lipase.

Further, the invention concerns the use of one or more isolated cultures or a composition comprising one or more bacterial cultures for reducing the amount of free fatty acids present on an item, wherein the bacteria culture is a strain of the genus Bacillus, Lysinibacillus or Pseudomonas.

In one embodiment of the invention, the bacterial culture has characteristics identical to one of the deposited strains, or a mixture thereof. The bacterial culture of the invention can comprise a mutant of at least one of the deposited strains, which mutant has all of the identifying characteristics of the corresponding deposited strain.

In one embodiment of the invention, the bacterial culture is one of the deposited strains or progeny thereof. The culture of the invention is capable of reducing the amount of free fatty acids from items comprising one or more free fatty acids. In one embodiment the items are laundry items such as textile or fabric. The item can be clothes, garnment, linen, table cloth or the like.

Free fatty acids that are odoreous are small chain free fatty acids with a chain length of from 2 to 10 carbon atoms. The inventors have found that the deposited strains are capable of reducing the amount of one or more free fatty acids, which free fatty acids has from 2 to 10 carbon atoms. The free fatty acids can be selected from the group consisting of formic acid, acetic acid, butyric acid, valeric acid, caproic acid, enanthic acid, caprylic acid, pelargonic acid and capric acid. Examples 2 and 3 shows that the bacterial culture according to the invention are capabable of reducing the amount of butyric and caproic acid derived from lipid stains when used together with a lipase in a wash cycle.

The invention further concerns a composition comprising one or more isolated bacteria cultures species selected from the group consisting of Bacillus subtilis, Bacillus subtilis subsp. subtilis, Lysinibacillus contaminans or Pseudomonas monteilii. The composition can be a detergent composition, a cleaning composition or a rinse aid or the composition can be an add-on composition used together with a conventional detergent composition, cleaning composition or rinsing aid. The composition can comprise one of the bacterial cultures according to the invention or the composition can comprise one or more of the bacteria cultures.

It is important that the bacterial culture is active during and/or after being used in a wash cycle. The composition therefore comprises bacteria culture as a vegetative, a dormant or a spore bacteria culture.

The composition can be a detergent or a cleaning composition that may in addition to the bacterial culture further comprise one or more detergent components selected from the group consisting of surfactants, builders, flocculating aid, chelating agents, dye transfer inhibitors, enzymes, enzyme stabilizers, enzyme inhibitors, catalytic materials, bleach activators, hydrogen peroxide, sources of hydrogen peroxide, preformed peracids, polymeric dispersing agents, clay soil removal/anti-redeposition agents, brighteners, suds suppressors, dyes, perfumes, structure elasticizing agents, fabric softeners, carriers, hydrotropes, builders and co-builders, fabric huing agents, anti-foaming agents, dispersants, processing aids, and/or pigments.

In one embodiment, the invention is directed to detergent compositions comprising an enzyme of the present invention in combination with one or more additional cleaning composition components. The choice of additional components is within the skill of the artisan and includes conventional ingredients, including the exemplary non-limiting components set forth below.

The choice of components may include, for textile care, the consideration of the type of textile to be cleaned, the type and/or degree of soiling, the temperature at which cleaning is to take place, and the formulation of the detergent product. Although components mentioned below are categorized by general header according to a particular functionality, this is not to be construed as a limitation, as a component may comprise additional functionalities as will be appreciated by the skilled artisan.

In one embodiment, the invention is directed to an ADW (Automatic Dish Wash) compositions comprising an enzyme of the present invention in combination with one or more additional ADW composition components. The choice of additional components is within the skill of the artisan and includes conventional ingredients, including the exemplary non-limiting components set forth below.

The composition can be a rinse aid such as a fabric softener comprising one or more bacterial cultures of the invention.

In one embodiment, the bacteria culture of the invention can be used in a fabric softener or a fabric conditioner. A fabric softener (also called fabric conditioner) is a chemical compound that is typically applied to laundry during the rinse cycle in a washing machine. Fabric softeners are available as solutions and solids, and may also be impregnated in dryer sheets used in a clothes dryer.

Fabric softeners coat the surface of a fabric with chemical compounds that are electrically charged, causing threads to “stand up” from the surface and thereby causing the fabric to feel softer. Cationic softeners bind by electrostatic attraction to the negatively charged groups on the surface of the fibers and neutralize their charge; the long aliphatic chains are then oriented towards the outside of the fiber, imparting lubricity.

The electrically conductive of fabric softener chemicals may also prevent buildup of static charge that can occur in clothes dryers. Other functions claimed by manufacturers include improvements of iron glide during ironing, increased stain resistance, reduction of wrinkling and pilling, and reduced drying time. Many softeners contain fragrances. Cationic fabric softeners are added during the rinse cycle rather than the wash cycle, as they can interfere with the cleaning action of detergents.

In addition to fabric softening chemicals, fabric softeners may include acids or bases for maintaining the optimal pH for absorption, silicone-based anti-foaming agents, emulsion stabilizers, fragrances, and colors.

Anionic softeners and antistatic agents can be, for example, salts of monoesters and diesters of phosphoric acid and the fatty alcohols. These are often used together with the conventional cationic softeners. Cationic softeners are incompatible with anionic surfactants used in detergents because they combine with them to form a solid precipitate. So, they must instead be added during the rinse cycle. Anionic softeners can be combined with anionic surfactants directly. Other anionic softeners can be based on smectite clays. Some compounds, such as ethoxylated phosphate esters, have softening, anti-static, and surfactant properties.

The composition can further comprise one or more enzymes. The enzymes can be comprised in the composition being a detergent or cleaning composition or a rinse aid, such as a fabric softener. The composition can further comprise one or more enzymes selected from the group consisting of amylases, arabinases, carbohydrases, cellulases, cutinases, DNases, galactanases, lipases, mannanases, oxidases, pectinases, proteases, and xylanases. In one embodiment the composition comprises a lipase.

In one embodiment of the invention, the composition comprises the bacterial cultures Bacillus subtilis and/or Pseudomonas monteilii. In one embodiment of the invention, the composition comprises the bacterial cultures Bacillus subtilis and/or Pseudomonas monteilii and a lipase. In one embodiment of the invention, the composition comprises the bacterial cultures Bacillus subtilis and/or Pseudomonas monteilii, a lipase and a surfactant. In one embodiment of the invention, the composition comprises the bacterial cultures Bacillus subtilis and/or Pseudomonas monteilii, a lipase and one or more detergent components. In one embodiment of the invention, the composition comprises the bacterial cultures Bacillus subtilis and/or Pseudomonas monteilii, a lipase and an anionic softener.

In one embodiment of the invention, the composition comprises the bacterial cultures Bacillus subtilis and/or Lysinibacillus contaminans. In one embodiment of the invention, the composition comprises the bacterial cultures Bacillus subtilis and/or Lysinibacillus contaminans and a lipase. In one embodiment of the invention, the composition comprises the bacterial cultures Bacillus subtilis and/or Lysinibacillus contaminans, a lipase and a surfactant. In one embodiment of the invention, the composition comprises the bacterial cultures Bacillus subtilis and/or Lysinibacillus contaminans, a lipase and one or more detergent components. In one embodiment of the invention, the composition comprises the bacterial cultures Bacillus subtilis and/or Lysinibacillus contaminans, a lipase and an anionic softener.

In one embodiment of the invention, the composition comprises the bacterial cultures Bacillus subtilis and/or Bacillus subtilis subsp. subtilis. In one embodiment of the invention, the composition comprises the bacterial cultures Bacillus subtilis and/or Bacillus subtilis subsp. subtilis and a lipase. In one embodiment of the invention, the composition comprises the bacterial cultures Bacillus subtilis and/or Bacillus subtilis subsp. subtilis, a lipase and a surfactant. In one embodiment of the invention, the composition comprises the bacterial cultures Bacillus subtilis and/or Bacillus subtilis subsp. subtilis, a lipase and one or more detergent components. In one embodiment of the invention, the composition comprises the bacterial cultures Bacillus subtilis and/or Bacillus subtilis subsp. subtilis, a lipase and an anionic softener.

In one embodiment of the invention, the composition comprises the bacterial cultures Bacillus subtilis, Lysinibacillus contaminans and/or Pseudomonas monteilii. In one embodiment of the invention, the composition comprises the bacterial cultures Bacillus subtilis, Lysinibacillus contaminans and/or Pseudomonas monteilii and a lipase. In one embodiment of the invention, the composition comprises the bacterial cultures Bacillus subtilis, Lysinibacillus contaminants and/or Pseudomonas monteilii, a lipase and a surfactant. In one embodiment of the invention, the composition comprises the bacterial cultures Bacillus subtilis, Lysinibacillus contaminans and/or Pseudomonas monteilii, a lipase and one or more detergent components. In one embodiment of the invention, the composition comprises the bacterial cultures Bacillus subtilis, Lysinibacillus contaminans and/or Pseudomonas monteilii, a lipase and an anionic softener.

In one embodiment of the invention, the composition comprises the bacterial culture with deposit number NRRL-B-67164 and/or NRRL B-50256. In one embodiment of the invention, the composition comprises the bacterial cultures with deposit number NRRL-B-67164 and/or NRRL B-50256 and a lipase. In one embodiment of the invention, the composition comprises the bacterial cultures with deposit number NRRL-B-67164 and/or NRRL B-50256, a lipase and a surfactant. In one embodiment of the invention, the composition comprises the bacterial cultures with deposit number NRRL-B-67164 and/or NRRL B-50256, a lipase and one or more detergent components. In one embodiment of the invention, the composition comprises the bacterial cultures with deposit numbers NRRL-B-67164 and/or NRRL B-50256, a lipase and an anionic softener.

In one embodiment of the invention, the composition comprises the bacterial culture with deposit number NRRL-B-67164 and/or NRRL-B-67160. In one embodiment of the invention, the composition comprises the bacterial cultures with deposit number NRRL-B-67164 and/or NRRL-B-67160 and a lipase. In one embodiment of the invention, the composition comprises the bacterial cultures with deposit number NRRL-B-67164 and/or NRRL-B-67160, a lipase and a surfactant. In one embodiment of the invention, the composition comprises the bacterial cultures with deposit number NRRL-B-67164 and/or NRRL-B-67160, a lipase and one or more detergent components. In one embodiment of the invention, the composition comprises the bacterial cultures with deposit numbers NRRL-B-67164 and/or NRRL-B-67160, a lipase and an anionic softener.

In one embodiment of the invention, the composition comprises the bacterial culture with deposit number NRRL-B-67164 and/or NRRL-B-67161. In one embodiment of the invention, the composition comprises the bacterial cultures with deposit number NRRL-B-67164 and/or NRRL-B-67161 and a lipase. In one embodiment of the invention, the composition comprises the bacterial cultures with deposit number NRRL-B-67164 and/or NRRL-B-67161, a lipase and a surfactant. In one embodiment of the invention, the composition comprises the bacterial cultures with deposit number NRRL-B-67164 and/or NRRL-B-67161, a lipase and one or more detergent components. In one embodiment of the invention, the composition comprises the bacterial cultures with deposit numbers NRRL-B-67164 and/or NRRL-B-67161, a lipase and an anionic softener.

In one embodiment of the invention, the composition comprises the bacterial culture with deposit number NRRL-B-67164, NRRL-B-67160 and/or NRRL B-50256. In one embodiment of the invention, the composition comprises the bacterial cultures with deposit number NRRL-B-67164 NRRL-B-67160 and/or NRRL B-50256 and a lipase. In one embodiment of the invention, the composition comprises the bacterial cultures with deposit number NRRL-B-67164, NRRL-B-67160 and/or NRRL B-50256, a lipase and a surfactant. In one embodiment of the invention, the composition comprises the bacterial cultures with deposit number NRRL-B-67164, NRRL-B-67160 and/or NRRL B-50256, a lipase and one or more detergent components. In one embodiment of the invention, the composition comprises the bacterial cultures with deposit numbers NRRL-B-67164, NRRL-B-67160 and/or NRRL B-50256, a lipase and an anionic softener.

In one embodiment of the invention, the composition comprises the bacterial culture with deposit number NRRL-B-67164, NRRL-B-67160, NRRL-B-67161, NRRL-B-67162, NRRL-B-67163 and/or NRRL B-50256. In one embodiment of the invention, the composition comprises the bacterial cultures with deposit number NRRL-B-67164, NRRL-B-67160, NRRL-B-67161, NRRL-B-67162, NRRL-B-67163 and/or NRRL B-50256 and a lipase. In one embodiment of the invention, the composition comprises the bacterial cultures with deposit number NRRL-B-67164, NRRL-B-67160, NRRL-B-67161, NRRL-B-67162, NRRL-B-67163 and/or NRRL B-50256, a lipase and a surfactant. In one embodiment of the invention, the composition comprises the bacterial cultures with deposit number NRRL-B-67164, NRRL-B-67160, NRRL-B-67161, NRRL-B-67162, NRRL-B-67163 and/or NRRL B-50256, a lipase and one or more detergent components. In one embodiment of the invention, the composition comprises the bacterial cultures with deposit numbers NRRL-B-67164, NRRL-B-67160, NRRL-B-67161, NRRL-B-67162, NRRL-B-67163 and/or NRRL B-50256, a lipase and an anionic softener.

The composition according to the invention is formulated so it is possible to obtain a concentration of each of the bacteria cultures in a wash liquor or water for rinsing is in the range of 1×10⁴ to 1×10¹² bacteria cells per 100 mL. In one embodiment, the concentration of each of the bacteria cultures in a wash liquor or water for rinsing is preferably in the range of 1×10⁴ to 1×10¹⁰ bacteria cells per 100 mL, in the range of 1×10⁴ to 1×10⁸ bacteria cells per 100 mL or in the range of 1×10⁴ to 1×10⁶ bacteria cells per 100 mL.

When the bacterial culture according to the invention is used for reducing the amount of free fatty acids present on an item, the amount of butyric acid and/or caproic acid is reduced with at least 50%, at least 55%, at least 60%, at least 65%, at least 70%, at least 75%, at least 80%, at least 85%, at least 90%, at least 95%, at least 96%, at least 97%, at least 98% or at least 99% when measured with Assay II. Example 2 demonstrates that the amount of butyric acid can be reduced by more than 99% after 21 hours. Example 3 shows that by using microbial spores the amount of butyric acid can be reduced by more than 99% after 31 hours.

The detergent or cleaning composition may be a liquid composition. For liquid detergent ingredients can be separated physically from each other by compartments in water dissolvable pouches. Thereby negative storage interaction between components can be avoided. Different dissolution profiles of each of the compartments can also give rise to delayed dissolution of selected components in the wash solution.

The detergent composition may take the form of a unit dose product. A unit dose product is the packaging of a single dose in a non-reusable container. It is increasingly used in detergents for laundry. A detergent unit dose product is the packaging (e.g., in a pouch made from a water soluble film) of the amount of detergent used for a single wash.

Pouches can be of any form, shape and material which is suitable for holding the composition, e.g., without allowing the release of the composition from the pouch prior to water contact. The pouch is made from water soluble film which encloses an inner volume. Said inner volume can be divided into compartments of the pouch. Preferred films are polymeric materials preferably polymers which are formed into a film or sheet. Preferred polymers, copolymers or derivates thereof are selected polyacrylates, and water soluble acrylate copolymers, methyl cellulose, carboxy methyl cellulose, sodium dextrin, ethyl cellulose, hydroxyethyl cellulose, hydroxypropyl methyl cellulose, malto dextrin, poly methacrylates, most preferably polyvinyl alcohol copolymers and, hydroxypropyl methyl cellulose (HPMC). Preferably the level of polymer in the film for example PVA is at least about 60%. Preferred average molecular weight will typically be about 20,000 to about 150,000. Films can also be a blend compositions comprising hydrolytically degradable and water soluble polymer blends such as polyactide and polyvinyl alcohol (known under the Trade reference M8630 as sold by Chris Craft In. Prod. Of Gary, Ind., US) plus plasticizers like glycerol, ethylene glycerol, Propylene glycol, sorbitol and mixtures thereof. The pouches can comprise a solid laundry cleaning composition or part components and/or a liquid cleaning composition or part components separated by the water soluble film. The compartment for liquid components can be different in composition than compartments containing solids (see, e.g., US 2009/0011970).

The choice of detergent components may include, for textile care, the consideration of the type of textile to be cleaned, the type and/or degree of soiling, the temperature at which cleaning is to take place, and the formulation of the detergent product. Although components mentioned below are categorized by general header according to a particular functionality, this is not to be construed as a limitation, as a component may comprise additional functionalities as will be appreciated by the skilled artisan.

The choice of additional components is within the skill of the artisan and includes conventional ingredients, including the exemplary non-limiting components set forth below.

The bacteria culture may be used during a wash comprising a wash liquor or a rinse comprising water and optionally a rinse aid. The bacterial culture may be used in a concentration of each of the bacteria cultures in a wash liquor or water for rinsing is in the range of 1×10⁴ to 1×10¹² bacteria cells per 100 mL. In one embodiment the concentration is preferably in the range of 1×104 to 1×10¹⁰ bacteria cells per 100 mL or in the range of 1×10⁴ to 1×10⁸ bacteria cells per 100 mL. In one embodiment of the invention the concentration of each of the bacteria cultures in the wash liquor or the water for rinsing is preferably in the range of 1×10⁴ to 1×10⁶ bacteria cells per 100 mL.

The bacteria culture may be used for reducing the amount of free fatty acids present on an item, wherein the item is a textile, a fabric, a dishware or a hard surface. The bacteria culture may be used during a wash cycle or a rinse cycle.

The invention further concerns a method for washing an item comprising the steps of:

a. Exposing the item to wash liquor;

b. Completing at least one wash cycle; and

c. Optionally rinsing the item with water optionally comprising a rinse aid,

wherein the wash liquor and/or the rinse aid comprise one or more bacteria cultures of the invention or a composition of the invention.

The item can be a textile, a fabric, a dishware or a hard surface. In one embodiment of the invention the hard surface is the interior of a laundry washing machine or an automated dish washing machine.

In one embodiment of the invention, the rinse aid is a fabric softener.

The bacterial culture may be used in a concentration of each of the bacteria cultures in a wash liquor or water for rinsing is in the range of 1×10⁴ to 1×10¹² bacteria cells per 100 mL. In one embodiment, the concentration is preferably in the range of 1×104 to 1×10¹⁰ bacteria cells per 100 mL or in the range of 1×10⁴ to 1×10⁸ bacteria cells per 100 mL. In one embodiment of the invention, the concentration of each of the bacteria cultures in the wash liquor or the water for rinsing is preferably in the range of 1×10⁴ to 1×10⁶ bacteria cells per 100 mL.

In one embodiment of the invention, the method for washing an item comprising the steps of:

a. Exposing the item to wash liquor;

b. Completing at least one wash cycle; and

c. Optionally rinsing the item with water optionally comprising a rinse aid,

wherein the wash liquor and/or the rinse aid comprise one or more bacteria cultures of the invention or a composition of the invention and wherein the bacteria culture reduces the amount of free fatty acids from items comprising free fatty acids. In one embodiment, the bacteria culture reduces the amount of free fatty acids having from 2 to 10 carbon atoms. In one embodiment, the bacteria culture reduces the amount of free fatty acid selected from the group consisting of formic acid, acetic acid, butyric acid, valeric acid, caproic acid, enanthic acid, caprylic acid, pelargonic acid and capric acid.

In one embodiment of the invention, the amount of butyric acid and/or caproic acid is reduced with at least 50%, at least 55%, at least 60%, at least 65%, at least 70%, at least 75%, at least 80%, at least 85%, at least 90%, at least 95%, at least 96%, at least 97%, at least 98% or at least 99% when measured with Assay II.

### Formulation of Detergent Products

The detergent composition of the invention may be in any convenient form, e.g., a bar, a homogenous tablet, a tablet having two or more layers, a pouch having one or more compartments, a regular or compact powder, a granule, a paste, a gel, or a regular, compact or concentrated liquid.

Pouches can be configured as single or multicompartments. It can be of any form, shape and material which is suitable for hold the composition, e.g., without allowing the release of the composition to release of the composition from the pouch prior to water contact. The pouch is made from water soluble film which encloses an inner volume. Said inner volume can be divided into compartments of the pouch. Preferred films are polymeric materials preferably polymers which are formed into a film or sheet. Preferred polymers, copolymers or derivates thereof are selected polyacrylates, and water soluble acrylate copolymers, methyl cellulose, carboxy methyl cellulose, sodium dextrin, ethyl cellulose, hydroxyethyl cellulose, hydroxypropyl methyl cellulose, malto dextrin, poly methacrylates, most preferably polyvinyl alcohol copolymers and, hydroxypropyl methyl cellulose (HPMC). Preferably the level of polymer in the film for example PVA is at least about 60%. Preferred average molecular weight will typically be about 20,000 to about 150,000. Films can also be of blended compositions comprising hydrolytically degradable and water soluble polymer blends such as polylactide and polyvinyl alcohol (known under the Trade reference M8630 as sold by MonoSol LLC, Indiana, USA) plus plasticisers like glycerol, ethylene glycerol, propylene glycol, sorbitol and mixtures thereof. The pouches can comprise a solid laundry cleaning composition or part components and/or a liquid cleaning composition or part components separated by the water soluble film. The compartment for liquid components can be different in composition than compartments containing solids: US 2009/0011970.

Detergent ingredients can be separated physically from each other by compartments in water dissolvable pouches or in different layers of tablets. Thereby negative storage interaction between components can be avoided. Different dissolution profiles of each of the compartments can also give rise to delayed dissolution of selected components in the wash solution.

A liquid or gel detergent, which is not unit dosed, may be aqueous, typically containing at least 20% by weight and up to 95% water, such as up to about 70% water, up to about 65% water, up to about 55% water, up to about 45% water, up to about 35% water. Other types of liquids, including without limitation, alkanols, amines, diols, ethers and polyols may be included in an aqueous liquid or gel. An aqueous liquid or gel detergent may contain from 0-30% organic solvent.

A liquid or gel detergent may be non-aqueous.

### Laundry Soap Bars

The bacteria culture of the invention may be added to laundry soap bars and used for hand washing laundry, fabrics and/or textiles. The term laundry soap bar includes laundry bars, soap bars, combo bars, syndet bars and detergent bars. The types of bar usually differ in the type of surfactant they contain, and the term laundry soap bar includes those containing soaps from fatty acids and/or synthetic soaps. The laundry soap bar has a physical form which is solid and not a liquid, gel or a powder at room temperature. The term solid is defined as a physical form which does not significantly change over time, i.e., if a solid object (e.g., laundry soap bar) is placed inside a container, the solid object does not change to fill the container it is placed in. The bar is a solid typically in bar form but can be in other solid shapes such as round or oval.

The laundry soap bar may contain one or more additional enzymes, protease inhibitors such as peptide aldehydes (or hydrosulfite adduct or hemiacetal adduct), boric acid, borate, borax and/or phenylboronic acid derivatives such as 4-formylphenylboronic acid, one or more soaps or synthetic surfactants, polyols such as glycerine, pH controlling compounds such as fatty acids, citric acid, acetic acid and/or formic acid, and/or a salt of a monovalent cation and an organic anion wherein the monovalent cation may be for example Na⁺, K⁺ or NH₄⁺ and the organic anion may be for example formate, acetate, citrate or lactate such that the salt of a monovalent cation and an organic anion may be, for example, sodium formate.

The laundry soap bar may also contain complexing agents like EDTA and HEDP, perfumes and/or different type of fillers, surfactants, e.g., anionic synthetic surfactants, builders, polymeric soil release agents, detergent chelators, stabilizing agents, fillers, dyes, colorants, dye transfer inhibitors, alkoxylated polycarbonates, suds suppressers, structurants, binders, leaching agents, bleaching activators, clay soil removal agents, anti-redeposition agents, polymeric dispersing agents, brighteners, fabric softeners, perfumes and/or other compounds known in the art.

The laundry soap bar may be processed in conventional laundry soap bar making equipment such as but not limited to: mixers, plodders, e.g., a two stage vacuum plodder, extruders, cutters, logo-stampers, cooling tunnels and wrappers. The invention is not limited to preparing the laundry soap bars by any single method. The premix of the invention may be added to the soap at different stages of the process. For example, the premix containing a soap, the bacteria culture of the invention, optionally one or more additional enzymes, a protease inhibitor, and a salt of a monovalent cation and an organic anion may be prepared and and the mixture is then plodded. The bacteria culture of the invention and optional additional enzymes may be added at the same time as the protease inhibitor for example in liquid form. Besides the mixing step and the plodding step, the process may further comprise the steps of milling, extruding, cutting, stamping, cooling and/or wrapping.

### Formulation of Bacteria in Co-Granule

The bacteria culture may be formulated as a granule for example as a co-granule that combines one or more enzymes. Each enzyme will then be present in more granules securing a more uniform distribution of enzymes in the detergent. This also reduces the physical segregation of the cultures due to different particle sizes. Methods for producing multi-enzyme co-granulates for the detergent industry are disclosed in the IP.com disclosure IPCOM000200739D.

Another example of formulation of enzymes by the use of co-granulates are disclosed in WO 2013/188331, which relates to a detergent composition comprising (a) a multi-enzyme co-granule; (b) less than 10 wt zeolite (anhydrous basis); and (c) less than 10 wt phosphate salt (anhydrous basis), wherein said enzyme co-granule comprises from 10 to 98 wt. % moisture sink component and the composition additionally comprises from 20 to 80 wt. % detergent moisture sink component. WO 2013/188331 also relates to a method of treating and/or cleaning a surface, preferably a fabric surface comprising the steps of (i) contacting said surface with the detergent composition as claimed and described herein in an aqueous wash liquor, (ii) rinsing and/or drying the surface.

The multi-enzyme co-granule may comprise a bactia culture of the invention and (a) one or more enzymes selected from the group consisting of first-wash lipases, cleaning cellulases, xyloglucanases, perhydrolases, peroxidases, lipoxygenases, laccases and mixtures thereof; and (b) one or more enzymes selected from the group consisting of hemicellulases, proteases, care cellulases, cellobiose dehydrogenases, xylanases, phospho lipases, esterases, cutinases, pectinases, mannanases, pectate lyases, keratinases, reductases, oxidases, phenoloxidases, ligninases, pullulanases, tannases, pentosanases, lichenases glucanases, arabinosidases, hyaluronidase, chondroitinase, amylases, and mixtures thereof.

### Assays and Detergent Compositions

### Detergent Compositions

The below mentioned detergent composition can be used in combination with the bacteria cultures of the invention.

### Biotex Black (Liquid)

5-15% Anionic surfactants, <5% Nonionic surfactants, perfume, enzymes, DMDM and hydantoin.

### Composition of Model Detergent B

Ingredients: 7% LAS, 3% AEOS/SLES, 6.6% NI, 5.5% soap (all percentages are w/w)

### Composition of Ariel Actilift (Liquid)

Ingredients: 5-15% Anionic surfactants; <5% Non-ionic surfactants, Phosphonates, Soap; Enzymes, Optical brighteners, Benzisothiazolinone, Methylisothiazolinone, Perfumes, Alpha-isomethyl ionone, Citronellol, Geraniol, Linalool.

### Composition of Ariel Sensitive White & Color, Liquid Detergent Composition

Ingredients: Aqua, Alcohol Ethoxy Sulfate, Alcohol Ethoxylate, Amino Oxide, Citrid Acid, C12-18 topped palm kernel fatty acid, Protease, Glycosidase, Amylase, Ethanol, 1,2 Propanediol, Sodium Formate, Calcium Chloride, Sodium hydroxide, Silicone Emulsion, Trans-sulphated EHDQ (the ingredients are listed in descending order).

### Composition of Ariel Actilift Colour&Style (Liquid)

Ingredients: 5-15% Anionic surfactants; <5% Non-ionic surfactants, Phosphonates, Soap; Enzymes, Perfumes, Benzisothiazolinone, Methylisothiazolinone, Alpha-isomethyl ionone, Butylphenyl methylpropional, Citronellol, Geraniol, Linalool.

### Composition of Persil Small & Mighty (Liquid)

Ingredients: 15-30% Anionic surfactants, Non-ionic surfacts, 5-15% Soap, <5% Polycarboxylates, Perfume, Phosphates, Optical Brighteners

Persil 2 in 1 with Comfort Passion Flower Powder

Sodium sulfate, Sodium carbonate, Sodium Dodecylbenzenesulfonate, Bentonite, Sodium Carbonate Peroxide, Sodium Silicate, Zeolite, Aqua, Citric acid, TAED, C12-15 Pareth-7, Stearic Acid, Parfum, Sodium Acrylic Acid/MA Copolymer, Cellulose Gum, Corn Starch Modified, Sodium chloride, Tetrasodium Etidronate, Calcium Sodium EDTMP, Disodium Anilinomorpholinotriazinyl-aminostilbenesulfonate, Sodium bicarbonate, Phenylpropyl Ethyl Methicone, Butylphenyl Methylpropional, Glyceryl Stearates, Calcium carbonate, Sodium Polyacrylate, Alpha-Isomethyl Ionone, Disodium Distyrylbiphenyl Disulfonate, Cellulose, Protease, Limonene, PEG-75, Titanium dioxide, Dextrin, Sucrose, Sodium Polyaryl Sulphonate, CI 12490, CI 45100, CI 42090, Sodium Thiosulfate, CI 61585.

### Persil Biological Powder

Sucrose, Sorbitol, Aluminum Silicate, Polyoxymethylene Melamine, Sodium Polyaryl Sulphonate, CI 61585, CI 45100, Lipase, Amylase, Xanthan gum, Hydroxypropyl methyl cellulose, CI 12490, Disodium Distyrylbiphenyl Disulfonate, Sodium Thiosulfate, CI 42090, Mannanase, CI 11680, Etidronic Acid, Tetrasodium EDTA.

### Composition of Fairy Non Bio (Liquid)

Ingredients: 15-30% Anionic Surfactants, 5-15% Non-Ionic Surfactants, Soap, Benzisothiazolinone, Methylisothiazolinone, Perfumes

### Composition of Model Detergent T (Powder)

Ingredients: 11% LAS, 2% AS/AEOS, 2% soap, 3% AEO, 15.15% sodium carbonate, 3% sodium silicate, 18.75% zeolite, 0.15% chelant, 2% sodium citrate, 1.65% AA/MA copolymer, 2.5% CMC and 0.5% SRP (all percentages are w/w).

### Composition of Model Detergent X (Powder)

Ingredients: 16.5% LAS, 15% zeolite, 12% sodium disilicate, 20% sodium carbonate, 1% sokalan, 35.5% sodium sulfate (all percentages are w/w).

### Gain Liquid, Original:

Ingredients: Water, Alcohol Ethoxysulfate, Diethylene Glycol, Alcohol Ethoxylate, Ethanolamine, Linear Alkyl Benzene Sulfonate, Sodium Fatty Acids, Polyethyleneimine Ethoxylate, Citric Acid, Borax, Sodium Cumene Sulfonate, Propylene Glycol, DTPA, Disodium Diaminostilbene Disulfonate, Dipropylethyl Tetramine, Sodium Hydroxide, Sodium Formate, Calcium Formate, Dimethicone, Amylase, Protease, Liquitint™, Hydrogenated Castor Oil, Fragrance

### Tide Liquid, Original:

Ingredients: Linear alkylbenzene sulfonate, propylene glycol, citric acid, sodium hydroxide, borax, ethanolamine, ethanol, alcohol sulfate, polyethyleneimine ethoxylate, sodium fatty acids, diquaternium ethoxysulfate, protease, diethylene glycol, laureth-9, alkyldimethylamine oxide, fragrance, amylase, disodium diaminostilbene disulfonate, DTPA, sodium formate, calcium formate, polyethylene glycol 4000, mannanase, Liquitint™ Blue, dimethicone.

### Tide Coldwater Liquid, Fresh Scent:

Water, alcoholethoxy sulfate, linear alkylbenzene sulfonate, diethylene glycol, propylene glycol, ethanolamine, citric acid, Borax, alcohol sulfate, sodium hydroxide, polyethyleneimine, ethoxylate, sodium fatty acids, ethanol, protease, Laureth-9, diquaternium ethoxysulfate, lauramine oxide, sodium cumene, sulfonate, fragrance, DTPA, amylase, disodium, diaminostilbene, disulfonate, sodium formate, disodium distyrylbiphenyl disulfonate, calcium formate, polyethylene glycol 4000, mannanase, pectinase, Liquitint™ Blue, dimethicone.

### Liquid Tide Plus Bleach Alternative™, Vivid White and Bright, Original and Clean Breeze:

Water, sodium alcoholethoxy sulfate, sodium alkyl sulfate, MEA citrate, linear alkylbenzene sulfonate, MEA salt, propylene glycol, diethylene glycol, polyethyleneimine ethoxylate, ethanol, sodium fatty acids, ethanolamine, lauramine oxide, borax, Laureth-9, DTPA, sodium cumene sulfonate, sodium formate, calcium formate, linear alkylbenzene sulfonate, sodium salt, alcohol sulfate, sodium hydroxide, diquaternium ethoxysulfate, fragrance, amylase, protease, mannanase, pectinase, disodium diaminostilbene disulfonate, benzisothiazolinone, Liquitint™ Blue, dimethicone, dipropylethyl tetraamine.

### Tide to go:

Deionized water, Dipropylene Glycol Butyl Ether, Sodium Alkyl Sulfate, Hydrogen Peroxide, Ethanol, Magnesium Sulfate, Alkyl Dimethyl Amine Oxide, Citric Acid, Sodium Hydroxide, Trimethoxy Benzoic Acid, Fragrance.

Tide Boost with Oxi:

Sodium bicarbonate, sodium carbonate, sodium percarbonate, alcohol ethoxylate, sodium chloride, maleic/acrylic copolymer, nonanoyloxy benzene sulfonate, sodium sulfate, colorant, diethylenetriamine pentaacetate sodium salt, hydrated aluminosilicate (zeolite), polyethylene glycol, sodium alkylbenzene sulfonate, sodium palmitate, starch, water, fragrance.

Ultra Tide with Downy Sun Blossom:

Water, sodium alcoholethoxy sulfate, MEA citrate, linear alkyl benzene sulfonate: sodium/MEA salts, propylene glycol, ethanol, diethylene glycol, polyethyleneimine propoxyethoxylate, polyethyleneimine ethoxylate, alcohol sulfate, dimethicone, fragrance, borax, sodium fatty acids, DTPA, protease, sodium bisulfite, disodium diaminostilbene disulfonate, amylase, castor oil, calcium formate, MEA, styrene acrylate copolymer, propanaminium propanamide, gluconase, sodium formate, Liquitint™ Blue.

### Tide Vivid White+Bright Powder, Original:

Sodium Carbonate, Sodium Aluminosilicate, Sodium Sulfate, Linear Alkylbenzene Sulfonate, Sodium Percarbonate, Nonanoyloxybenzenesulfonate, Alkyl Sulfate, Water, Silicate, Sodium Polyacrylate Ethoxylate, Polyethylene Glycol 4000, Fragrance, DTPA, Palmitic Acid, Protease, Disodium Diaminostilbene Disulfonate, Silicone, FD&C Blue 1, Cellulase, Alkyl Ether Sulfate.

### HEY SPORT TEX WASH Detergent

Aqua, dodecylbenzenesulfonsaure, laureth-11, peg-75 lanolin, propylene glycol, alcohol denat, potassium soyate, potassium hydroxide, disodium cocoamphodiacetate, ethylendiamine triacetate cocosalkyl acetamide, parfum, zinc ricinoleate, sodium chloride, benzisothiazolinone, methylisothiazolinone, ci 16255, benzyl alcohol.

The products named Tide, Ariel, Gain and Fairy are commercially available products supplied by Procter & Gamble. The products named Persil are commercially available products supplied by Unilever and Henkel. The products named Hey Sport are commercially available products supplied by Hey Sport.

### Fabric Softeners

The below mentioned fabric softeners can be used in combination with the bacteria cultures of the invention.

### Ultra Downy® Liquid—April Fresh

Water, diethyl ester dimethyl ammonium chloride, Fragrance, Starch, Ammonium chloride, Calcium chloride, Formic acid, Polydimethylsiloxane, Liquitint™, Benzisothiazolinone, diethylenetriamine pentaacetate (sodium salt).

### Medias and Solutions

### Minimal Salts Broth (MSB) Preparation

### Preparation of Solution A

Dibasic sodium phosphate solution (1M) was prepared using 142 g/L dibasic sodium phosphate (Sigma #30435-500 g, Lot SZBE0760V). Monobasic potassium phosphate solution (1 M) was prepared using 136 g/L monobasic potassium phosphate (Fisher # P285-3, Lot 110377). A mixture of these solutions was prepared by combining 700 mL of 1 M dibasic sodium phosphate solution with 300 mL 1 M monobasic potassium phosphate solution resulting in a solution with pH=7.3.

### Preparation of Solution B: Hunter's Concentrated Base

A solution of deionized water (700 mL), 20 g/L nitrilotriacetic acid NTA-free acid (Fisher # BP2670-100, Lot 126106), 14 g/L potassium hydroxide (Fisher # P251-3, Lot 136256), 59.3 g/L magnesium sulfate heptahydrate (CAS #10034-99-8), and 6.67 g/L calcium chloride (Sigma # C3881, Batch 088K0060) was prepared. Additional potassium hydroxide pellets are added until the precipitate dissolves after which 0.0185 g/L ammonium molybdate tetrahydrate (Sigma # M1019-100 g, Lot SLBD0365V), and 0.195 g/L ferrous sulfate heptahydrate (Sigma #31,007-7, Lot 05208KX) was added. The pH was adjusted to 6.8 using 10 N potassium hydroxide.

### Preparation of Metals “44” Solution

A solution of deionized water (800 mL), 2.5 g ethylenediaminetetraacetic acid (EDTA) (Acros #118432500, Lot A0336458), 10.95 g/L zinc sulfate (Fisher # Z68-500, Lot 122848A), 5 g/L iron II sulfate heptahydrate (Sigma #31,007-7, Lot 05208KX), 1.54 g/L manganese sulfate (Fisher # M113-500, Lot 121598), 0.392 g/L copper II sulfate (Sigma Cat #451657), 0.250 g/L cobalt (II) nitrate hexahydrate (Sigma Cat #239267), and 0.177 g/L sodium tetraborate (Sigma # B-9876, Lot 35H3556) was prepared. The pH was adjusted to 6.8 using 10 N potassium hydroxide at which point the solution obtained a green coloration.

### Preparation of Solution C

A 20% solution of ammonium sulfate (Fisher # A702-500, Lot 128025) was prepared in deionized water.

### Preparation of MSB Solution

The MSB solution was prepared by mixing 40 mL solution A, 10 mL solution B, and 5 mL solution C into 945 mL de-ionized water. Disposable vacuum sterilization filter units (Fisher #097403A) were used to sterilize the medium.

### Preparation of Schaffer's Medium

Schaffer's medium was prepared by mixing 8 g/L nutrient broth, 2.0212 g/L potassium chloride (Sigma # P3911), 0.492 g/L magnesium sulfate heptahydrate, 0.236 g/L calcium nitrate tetrahydrate (Mallinckrodt 4236, Lot #4236T04586), 0.0197 g/L manganese (II) chloride tetrahydrate (Fisher AC20589, Lot #955894), and 0.000278 g/L iron (II, III) oxide (Strem Chemicals #93-2616, Lot #20083200). The medium was autoclaved for 20 min using liquid cycle. The pH was adjusted to 6.9±0.2 at 25° C.

### Preparation of Bacillus Spores

Schaffer's medium (10 mL in a 250 mL baffled Erlenmeyer flask) was inoculated with 1 colony of vegetative cells taken from a Remel Plate Count Agar streak plate and was agitated at 200 rpm for 7 days at 30° C. during which the majority of the vegetative cell sporulated. Samples where heated to 80° C. for 10 minutes, centrifuged at 12,000 rpm for 3 minutes at 4° C., the aqueous phase was removed and replaced with sterile de-ionized water, and centrifuged at 12,000 rpm for 3 minutes. This was repeated thrice. Serial dilutions were performed on the spore solution by mixing 900 μL phosphate buffer solution (Weber #3127-29) and 100 μL of spore solution to a 1500 μL micro-centrifuge tube (Fisher Cat #87003-294). This −1 dilution was vortexed for 10 seconds at maximum setting. From this solution, 100 μL was further diluted into 900 μL phosphate buffer solution in a 1500 μL micro-centrifuge tube and vortexed for 10 seconds at the maximum setting (−2 dilution). This was continued until the −8 dilution was achieved. Standard method agar plates (Smith River Biologicals) were obtained and 100 μL of the aforementioned sample dilutions were applied to plate and spread using a lazy L spreader (Fisher Cat #14-373-76). The plates were incubated at 23° C. for 72 hours and the CFU/mL counted manually and recorded.

### Preparation of Microbial Vegetative Cells

Tryptic soy broth (10 mL) was added to test tubes and was inoculated with 1 colony of vegetative cells taken from a Remel Plate Count Agar streak plate and was agitated at 200 rpm for 18 hours at 30° C. Serial dilutions were performed on the microbial solution by mixing 900 μL phosphate buffer solution (Weber #3127-29) and 100 μL of microbial solution to a 1500 μL micro-centrifuge tube (Fisher Cat #87003-294). This −1 dilution was vortexed for 10 seconds at maximum setting. From this solution, 100 μL was further diluted into 900 μL phosphate buffer solution in a 1500 μL micro-centrifuge tube and vortexed for 10 seconds at the maximum setting (−2 dilution). This was continued until the −8 dilution was achieved. Standard method agar plates (Smith River Biologicals) were obtained and 100 μL of the aforementioned sample dilutions were applied to plate and spread using a lazy L spreader (Fisher Cat #14-373-76). The plates were incubated at 23° C. for 72 hours and the CFU/mL counted manually and recorded.

### Assays

### Assay I: Wash Assay

### Textile Washing Procedure

One hundred milliliters of 23° C. millipore filtered water (Millipore Simplicity) was added to 237 mL bottles (Fisher #03-313-15D) to which 4.4 g/L Model B Detergent, 0.1% Lipex™ 100 L (Novoymes A/S, not used in blank), 5-5.5×5.5 cm cotton swatches (WFK Testgewebe code 10A), and 1-5×5 cm CS10 swatch (WFK Testgewebe), were added to each bottle. These bottles were shaken on a wrist action shaker (Burrell Model 75) set at a 45° angle with an agitation setting of 10 for 20 minutes. The washing liquid was decanted, cloth removed and placed into a 30 mL sterile syringe (Fisher #14-829-48), and the plunger depressed 22 mL (to the 8 mL marking) to drain excess wash liquid. All cloth was removed from syringe, straightened, and placed back into the original plastic bottle with the CS10 uppermost.

### Rinsing and Inoculation of Textiles

Minimal salts broth (MSB) was thermally equilibrated to 23° C. Minimal salts broth (100 mL), 0.1% lipase 100 L, and 1 mL of vegetative culture grown in tryptic soy broth (or desired concentration of microbial spore preparation as shown in table 1) was added to 237 mL bottles containing the washed cotton and CS10 swatch. Blank control samples were prepared following the same procedure without Lipex or microbial addition to demonstrate the lack of butyric or caproic acid formation in their absence. The bottles were shaken on a wrist action shaker set to a 45° angle with an agitation setting of 10 for 20 minutes. The MSB solution was decanted and discarded. The cloth was removed from each bottle and placed into a sterile 30 mL syringe and the plunger depressed 22 mL (to the 8 mL marking) to drain excess wash liquid. The CS10 swatch was immediately placed into a 20 mL GC vial (Alpha MOS part #202-0050) and crimp sealed using a GC crimp cap (Alpha MOS part #202-0060). The samples were incubated at 23° C. and analyzed by GC/FID (Assay II) approximately every 10 hours.

TABLE 1
Spore concentrations used for present work

| Deposit | | Inoculum | Standard |
|---|---|---|---|
| Number | Strain ID | CFU/mL | Deviation |
| NRRL B-50256 | Pseudomonas monteilii | N/A | N/A |
| NRRL-B-67160 | Lysinibacillus | 4.90e6 | 5.29e5 |
| | contaminans | | |
| NRRL-B-67161 | Bacillus subtilis subsp. | 1.27e10 | 2.60e9 |
| | subtilis | | |
| NRRL-B-67162 | Bacillus subtilis | 5.77e5 | 4.80e5 |
| NRRL-B-67163 | Bacillus subtilis | 5.90e7 | 6.08e6 |
| NRRL-B-67164 | Bacillus subtilis | 1.33e6 | 5.77e5 |

### Assay II: GC/GID Analysis

As used herein, the term “Gas Chromatography” or “GC” refers to an analytical analysis technique in which a sample is injected, vaporized, passed through a column using a carrier gas which separates the individual components of a sample. Each component produces a specific peak based on its retention time within the column. The retention time is the elapsed time between injection into and the elution from the column.

As used herein, the term “Flame Ionization Detection” or “FID” refers to a specific type of detection method in which ions are generated by the combustion of organic compounds in a hydrogen flame. Component detection is accomplished by ion attraction to a collector plate inducing a current corresponding to the proportion of reduced carbon atoms. This is proportional to the concentration of organic compounds in the sample.

As used herein, the term “Gas Chromatography/Flame Ionized Detection” or “GC/FID” refers to the combination of GC and FID in which a sample passes through the GC for separation and passed through an FID for detection.

### GC/FID

Samples were analyzed by Gas Chromatography (GC) using a Shimadzu GC-2010 with 50/30 μm divinylbenzene/carboxen on polydimethylsiloxane (PDMS) Solid Phase Micro Extraction Fiber (SPME Fiber (Sigma #57299U). The column used was a 30.0 m DB-FFAP (Agilent Part #123-3232E, Serial # USB345626H) with an inner diameter of 0.32 and a film thickness of 0.25 μm. The data was processed using GC Solution Version 2.40 software.

### Column Settings

Analysis conditions for the GC column were as follows: column temperature 80.0° C., equilibration time 0.5 min, column oven temperature program set to 80° C. for 1 minute and then increased to 200° C. at a rate of 20° C./min and held at 200° C. for 4 minutes resulting in a run time of 11 minutes. Sample delivery was accomplished using a CTC Analytics CombiPAL auto-sampler.

### FID Settings

Analysis conditions for the FID were as follows: temperature set to 260.0° C., sampling rate of 80 msec, makeup gas Helium at 30 mL/min, Hydrogen flow 50.0 mL/min, and air flow of 400 mL/min.

### Auto-Sampler Settings

The following auto sampler settings were used: SPME cycle, fiber syringe, 5 min pre-incubation at 35° C., no agitation, vial penetration of 22.0 mm, 5 min extraction time, 54.0 mm injection penetration, 10 min desorption, no fiber bakeout, and a total runtime of 11 min.

### EXAMPLES

### Materials and Methods

As used herein, the term “strains” or “microbial cell”, microbial spore” or microbe refers to the microorganisms shown in table 1 used during the present work.

### Example 1

### Identification, Characterization and Deposit of the Biological Material

The following biological materials were deposited under the terms of the Budapest Treaty at the Agricultural Research Service Culture Collection (NRRL) of the U.S. Department of Agriculture located at 1815 North University Street, Peoria, Ill. 61604 U.S.A., and given the following accession numbers:

TABLE 1
Deposit of Biological Material

| Identification | Accession Number | Date of Deposit |
|---|---|---|
| Pseudomonas monteilii | NRRL B-50256 | Feb. 18, 2009 |
| Lysinibacillus contaminans | NRRL-B-67160 | Dec. 10, 2015 |
| Bacillus subtilis subsp. subtilis | NRRL-B-67161 | Dec. 10, 2015 |
| Bacillus subtilis | NRRL-B-67162 | Dec. 10, 2015 |
| Bacillus subtilis | NRRL-B-67163 | Dec. 10, 2015 |
| Bacillus subtilis | NRRL-B-67164 | Dec. 10, 2015 |

The strains have been deposited under conditions that assure that access to the culture will be available during the pendency of this patent application to one determined by foreign patent laws to be entitled thereto. The deposits represent a substantially pure culture of the deposited strains. The deposits are available as required by foreign patent laws in countries wherein counterparts of the subject application or its progeny are filed. However, it should be understood that the availability of a deposit does not constitute a license to practice the subject invention in derogation of patent rights granted by governmental action.

### Example 2

### Microbial Reduction of Butyric and Caproic Acids Using Vegetative Cell Inoculum

### Introduction

Lipases can generate objectionable fatty acid odors resultant of lipid degradation to short-chain volatile fatty acids. The washing and subsequent rinsing of lipid containing CS10 swatches in the presence of lipase as described in assay I resulted in the formation of butyric and caproic acids. Application of vegetative microbial cells can cause a reduction of these acids.

In the present example, CS10 swatches were washed according to Assay I with Lipex™ and microbes (NRRL B-50256, NRRL B-6760, NRRL B-6762). A blank sample of CS10, in which neither lipase nor microbes were added, was included to demonstrate that butyric and caproic acids were not created in the absence of lipase and microbes.

TABLE 2
GC/FID Peak area values for butyric acid after vegetative treatment as measured according to Assay II.
Relative Intensity of Butyric Acid After Vegetative Microbe Treatment

| Incubation | Blank | | Lipex | | NRRL B-50256 | | NRRL B-67160 | | NRRL B-67162 | |
|---|---|---|---|---|---|---|---|---|---|---|
| Time | | Standard | | Standard | | Standard | | Standard | | Standard |
| (hh:mm:ss) | Average | Deviation | Average | Deviation | Average | Deviation | Average | Deviation | Average | Deviation |
| 0:00:00 | 0.00 | 0.00 | 527.67 | 302.08 | 160.87 | 78.09 | 80.07 | 93.93 | 206.57 | 204.92 |
| 10:35:26 | 0.00 | 0.00 | 515.87 | 328.98 | 17.13 | 14.96 | 489.47 | 692.00 | 339.67 | 333.31 |
| 21:10:57 | 0.00 | 0.00 | 2028.43 | 905.97 | 14.50 | 25.11 | 2344.37 | 3680.61 | 910.57 | 848.30 |
| 31:46:23 | 0.00 | 0.00 | 4860.37 | 830.76 | 41.03 | 48.19 | 2749.37 | 2888.31 | 2328.47 | 1926.04 |
| 42:21:56 | 7.67 | 13.28 | 5959.20 | 799.26 | 160.83 | 174.03 | 2914.17 | 2300.26 | 1906.30 | 1121.71 |
| 52:57:30 | 13.33 | 11.64 | 7430.87 | 1046.62 | 470.07 | 398.14 | 3128.97 | 1298.60 | 2485.80 | 1258.31 |
| 63:33:05 | 11.83 | 20.50 | 8047.00 | 1509.12 | 813.13 | 291.28 | 3479.80 | 1405.94 | 2679.87 | 1224.07 |
| 74:08:37 | 29.57 | 36.81 | 8954.80 | 1879.32 | 1449.03 | 363.91 | 3596.03 | 1654.41 | 2990.77 | 1063.61 |
| 84:44:14 | 32.80 | 31.20 | 8983.30 | 3024.26 | 1953.97 | 1077.69 | 3398.93 | 1535.21 | 3100.70 | 1024.95 |
| 95:19:40 | 59.77 | 55.72 | 10493.43 | 2040.82 | 2400.70 | 955.26 | 3793.97 | 2044.93 | 2685.93 | 768.43 |
| 105:55:14 | 59.97 | 51.84 | 10787.47 | 1873.26 | 2721.70 | 1888.43 | 3746.17 | 1704.10 | 2537.30 | 1100.13 |

TABLE 3
Percent butyric acid reduction after vegetative cell treatment
as measured according to Assay II.
Percent Butyric Acid Reduced as Compared to Lipase Control After
Microbial Treatment

| Incubation | | | | |
|---|---|---|---|---|
| Time | | NRRL B- | NRRL B- | NRRL B- |
| (hh:mm:ss) | Blank | 50256 | 67160 | 67162 |
| 0:00:00 | 100.00 | 69.51 | 84.83 | 60.85 |
| 10:35:26 | 100.00 | 96.68 | 5.12 | 34.16 |
| 21:10:57 | 100.00 | 99.29 | −15.58 | 55.11 |
| 31:46:23 | 100.00 | 99.16 | 43.43 | 52.09 |
| 42:21:56 | 99.87 | 97.30 | 51.10 | 68.01 |
| 52:57:30 | 99.82 | 93.67 | 57.89 | 66.55 |
| 63:33:05 | 99.85 | 89.90 | 56.76 | 66.70 |
| 74:08:37 | 99.67 | 83.82 | 59.84 | 66.60 |
| 84:44:14 | 99.63 | 78.25 | 62.16 | 65.48 |
| 95:19:40 | 99.43 | 77.12 | 63.84 | 74.40 |
| 105:55:14 | 99.44 | 74.77 | 65.27 | 76.48 |

TABLE 4
GC/FID Peak area values for caproic acid after vegetative treatment as measured according to Assay II.
Relative Intensity of Caproic Acid After Vegetative Microbe Treatment

| Incubation | Blank | | Lipex | | NRRL B-50256 | | NRRL B-67160 | | NRRL B-67162 | |
|---|---|---|---|---|---|---|---|---|---|---|
| Time | | Standard | | Standard | | Standard | | Standard | | Standard |
| (hh:mm:ss) | Average | Deviation | Average | Deviation | Average | Deviation | Average | Deviation | Average | Deviation |
| 0:00:00 | 50.43 | 19.74 | 1,003.90 | 528.56 | 698.30 | 313.15 | 185.20 | 206.53 | 383.77 | 354.92 |
| 10:35:26 | 4.70 | 8.14 | 1,117.83 | 662.23 | 323.43 | 170.54 | 1,103.13 | 1,568.73 | 763.30 | 722.29 |
| 21:10:57 | 29.50 | 9.20 | 4,454.53 | 1,709.68 | 160.43 | 81.61 | 1,186.93 | 1,247.44 | 2,133.10 | 2,131.20 |
| 31:46:23 | 26.67 | 23.31 | 10,043.63 | 1,536.80 | 157.67 | 101.36 | 5,955.43 | 5,904.61 | 5,108.67 | 4,245.05 |
| 42:21:56 | 63.87 | 21.42 | 11,398.87 | 697.65 | 265.83 | 198.85 | 6,118.20 | 4,241.37 | 4,209.27 | 2,238.66 |
| 52:57:30 | 65.37 | 46.19 | 13,417.10 | 904.11 | 508.23 | 322.31 | 6,445.53 | 1,907.04 | 5,198.63 | 2,400.58 |
| 63:33:05 | 83.60 | 38.27 | 13,765.57 | 1,830.02 | 806.47 | 298.13 | 7,066.90 | 2,053.01 | 5,420.97 | 2,224.87 |
| 74:08:37 | 94.47 | 66.16 | 14,830.17 | 2,719.92 | 1,233.87 | 548.68 | 6,940.47 | 2,058.39 | 5,902.73 | 1,750.36 |
| 84:44:14 | 90.53 | 63.98 | 15,121.37 | 3,549.81 | 1,683.27 | 1,343.62 | 6,578.00 | 2,028.94 | 5,913.63 | 1,600.43 |
| 95:19:40 | 103.07 | 96.96 | 16,000.10 | 2,363.55 | 1,826.77 | 1,274.87 | 6,954.40 | 2,599.39 | 5,296.67 | 985.48 |
| 105:55:14 | 114.80 | 59.37 | 16,254.90 | 2,444.61 | 2,029.30 | 1,927.52 | 7,037.37 | 2,112.57 | 5,607.13 | 1,477.09 |

TABLE 5
Percent caproic acid reduction after vegetative cell treatment
as measured according to Assay II.
Percent Caproic Acid Reduced as Compared to Lipase Control After
Microbial Treatment

| Incubation | | | | |
|---|---|---|---|---|
| Time | | NRRL B- | NRRL B- | NRRL B- |
| (hh:mm:ss) | Blank | 50256 | 67160 | 67162 |
| 0:00:00 | 94.98 | 30.44 | 81.55 | 61.77 |
| 10:35:26 | 99.58 | 71.07 | 1.32 | 31.72 |
| 21:10:57 | 99.34 | 96.40 | 73.35 | 52.11 |
| 31:46:23 | 99.73 | 98.43 | 40.70 | 49.14 |
| 42:21:56 | 99.44 | 97.67 | 46.33 | 63.07 |
| 52:57:30 | 99.51 | 96.21 | 51.96 | 61.25 |
| 63:33:05 | 99.39 | 94.14 | 48.66 | 60.62 |
| 74:08:37 | 99.36 | 91.68 | 53.20 | 60.20 |
| 84:44:14 | 99.40 | 88.87 | 56.50 | 60.89 |
| 95:19:40 | 99.36 | 88.58 | 56.54 | 66.90 |
| 105:55:14 | 99.29 | 87.52 | 56.71 | 65.50 |

### Results and Discussion

All of the investigated microbes were capable of reducing butyric and caproic acid levels generated by lipase action in the laundry environment when applied as vegetative cells (Table 3). NRRL B50256 demonstrated a butyric acid reduction of 99.29% at 21.2 hours and upon completion of the study still retained a 74.77% reduction. The percent reduction of butyric acid improved from 31.8 to 105.9 hours when treated with NRRL-B-67160 or NRRL-B-67162 reaching final reduction levels of 65.27% and 76.48% respectively.

With respect to caproic acid reduction (Table 5), NRRL B50256 demonstrated a caproic acid reduction of 98.43% after 31.8 hours and continued to reduce 87.52% of caproic acid by 105.9 hours. NRRL-B-67160 achieved a 56.71% percent reduction of caproic acid by 105.9 hours. NRRL-B-67162 had reduced 65.5% of caproic acid by 105.9 hours.

### Example 3

### Reduction of Butyric and Caproic Acid Using Microbial Spores

### Introduction

Lipases can generate objectionable fatty acid odors resultant of lipid degredation to short-chain volatile fatty acids. The washing and subsequent rinsing of lipid containing CS10 swatches in the presence of lipase as described in assay I result in the formation of butyric and caproic acids. Application of microbial spores, which germinate into vegetative cells, can result in the reduction of these acids.

In the present example, CS10 swatches were washed according to Assay I with lipase (Lipex™, available from Novozymes A/S) and microbial spores (NRRL B-50256, NRRL B-6761, NRRL B-6763, NRRL B-6764). A blank sample (CS10) which contained neither lipase nor microbial spore was included to demonstrate that butyric and caproic acids were not created in the absence of lipase and microbial spores.

TABLE 6
GC/FID Peak area values for butyric acid after microbial spore treatment as measured according to Assay II.
Relative Intensity of Butyric Acid After Microbial Spore Treatment

| Incubation | Blank | | Lipex | | NRRL B-50256 | | NRRL B-67161 | | NRRL B-67163 | | NRRL B-67164 | |
|---|---|---|---|---|---|---|---|---|---|---|---|---|
| Time | Percent | Standard | Percent | Standard | Percent | Standard | Percent | Standard | Percent | Standard | Percent | Standard |
| (hh:mm:ss) | Average | Deviation | Average | Deviation | Average | Deviation | Average | Deviation | Average | Deviation | Average | Deviation |
| 0:00:00 | 31.33 | 28.41 | 337.00 | 237.12 | 280.83 | 153.69 | 65.50 | 31.63 | 6.67 | 11.55 | 55.83 | 50.61 |
| 10:35:25 | 5.03 | 8.72 | 108.23 | 67.02 | 44.10 | 42.63 | 119.00 | 72.54 | 22.63 | 19.62 | 125.03 | 113.84 |
| 21:11:07 | 18.60 | 20.03 | 358.77 | 338.01 | 17.40 | 16.04 | 147.27 | 86.15 | 113.47 | 98.56 | 221.73 | 140.68 |
| 31:46:37 | 0.00 | 0.00 | 1008.87 | 655.24 | 2.63 | 4.56 | 674.33 | 294.79 | 351.80 | 137.49 | 283.03 | 177.58 |
| 42:22:11 | 6.13 | 7.50 | 1188.20 | 1087.90 | 27.60 | 18.76 | 1290.40 | 59.51 | 410.33 | 226.43 | 398.67 | 105.49 |
| 52:57:43 | 14.53 | 17.81 | 1923.80 | 663.18 | 309.70 | 322.53 | 1460.90 | 658.97 | 486.23 | 118.01 | 277.87 | 158.77 |
| 63:33:13 | 13.37 | 15.53 | 2129.80 | 904.53 | 415.67 | 296.55 | 1756.33 | 583.69 | 647.47 | 89.80 | 322.93 | 317.19 |
| 74:08:34 | 6.17 | 10.68 | 2265.27 | 813.05 | 637.60 | 634.65 | 2005.60 | 556.10 | 556.90 | 101.38 | 306.63 | 375.71 |
| 84:44:04 | 11.53 | 10.21 | 2303.83 | 981.10 | 962.27 | 912.07 | 2486.27 | 1026.94 | 475.30 | 108.05 | 344.30 | 407.59 |

TABLE 7
Percent butyric acid reduction after spore treatment
Percent Butyric Acid Reduction Using Microbial Spores

| Incubation | | | | | |
|---|---|---|---|---|---|
| Time | | NRRL B- | NRRL B- | NRRL B- | NRRL B- |
| (hh:mm:ss) | Blank | 50256 | 67161 | 67163 | 67164 |
| 0:00:00 | 90.70 | 16.67 | 80.56 | 98.02 | 83.43 |
| 10:35:25 | 95.35 | 59.25 | −9.95 | 79.09 | −15.52 |
| 21:11:07 | 94.82 | 95.15 | 58.95 | 68.37 | 38.20 |
| 31:46:37 | 100.00 | 99.74 | 33.16 | 65.13 | 71.95 |
| 42:22:11 | 99.48 | 97.68 | −8.60 | 65.47 | 66.45 |
| 52:57:43 | 99.24 | 83.90 | 24.06 | 74.73 | 85.56 |
| 63:33:13 | 99.37 | 80.48 | 17.54 | 69.60 | 84.84 |
| 74:08:34 | 99.73 | 71.85 | 11.46 | 75.42 | 86.46 |
| 84:44:04 | 99.50 | 58.23 | −7.92 | 79.37 | 85.06 |

TABLE 8
GC/FID Peak area values for caproic acid after microbial spore treatment as measured according to Assay II.
Relative Intensity of Caproic Acid After Microbial Spore Treatment

| Incubation | Blank | | Lipex | | NRRL B-50256 | | NRRL B-67161 | | NRRL B-67163 | | NRRL B-67164 | |
|---|---|---|---|---|---|---|---|---|---|---|---|---|
| Time | Percent | Standard | Percent | Standard | Percent | Standard | Percent | Standard | Percent | Standard | Percent | Standard |
| (hh:mm:ss) | Average | Deviation | Average | Deviation | Average | Deviation | Average | Deviation | Average | Deviation | Average | Deviation |
| 0:00:00 | 114.40 | 114.85 | 504.00 | 330.90 | 561.10 | 365.77 | 110.67 | 50.82 | 43.53 | 59.71 | 100.57 | 97.29 |
| 10:35:25 | 25.97 | 22.58 | 247.23 | 205.95 | 584.80 | 441.47 | 263.90 | 181.68 | 72.50 | 35.40 | 301.47 | 220.71 |
| 21:11:07 | 13.57 | 23.50 | 925.57 | 755.80 | 213.30 | 150.40 | 343.63 | 140.60 | 277.10 | 161.28 | 493.90 | 368.64 |
| 31:46:37 | 14.57 | 25.23 | 2,291.03 | 1,476.54 | 118.57 | 112.27 | 1,537.60 | 484.61 | 787.53 | 176.47 | 715.90 | 394.05 |
| 42:22:11 | 16.13 | 18.55 | 2,664.50 | 2,200.07 | 115.70 | 69.75 | 2,584.90 | 326.88 | 899.30 | 425.55 | 927.73 | 227.08 |
| 52:57:43 | 43.20 | 14.91 | 4,134.83 | 1,168.08 | 386.93 | 433.61 | 3,134.30 | 1,126.61 | 1,082.20 | 234.04 | 732.50 | 353.45 |
| 63:33:13 | 27.70 | 2.51 | 4,255.33 | 1,328.58 | 484.10 | 346.25 | 3,819.70 | 1,168.81 | 1,472.47 | 294.19 | 833.17 | 699.01 |
| 74:08:34 | 16.20 | 28.06 | 4,366.47 | 1,371.11 | 687.63 | 618.96 | 4,135.33 | 1,138.18 | 1,270.60 | 115.18 | 698.43 | 687.25 |
| 84:44:04 | 26.67 | 26.31 | 4,557.37 | 1,761.18 | 740.10 | 603.71 | 4,914.97 | 1,754.20 | 1,252.87 | 453.28 | 794.83 | 925.80 |

TABLE 9
Percent caproic acid reductioin after spore treatment
as measured according to Assay II.
Percent Caproic Acid Using Microbial Reduction

| Incubation | | | | | |
|---|---|---|---|---|---|
| Time | | NRRL B- | NRRL B- | NRRL B- | NRRL B- |
| (hh:mm:ss) | Blank | 50256 | 67163 | 67164 | 67164 |
| 0:00:00 | 77.30 | −11.33 | 78.04 | 91.36 | 80.05 |
| 10:35:25 | 89.50 | −136.54 | −136.40 | 70.68 | −21.94 |
| 21:11:07 | −98.53 | 76.95 | 62.87 | 70.06 | 46.64 |
| 31:46:37 | 99.36 | 94.82 | 32.89 | 65.63 | 68.75 |
| 42:22:11 | 99.39 | 95.66 | 2.99 | 66.25 | 65.18 |
| 52:57:43 | 98.96 | 90.64 | 24.20 | 73.83 | 82.28 |
| 63:33:13 | 99.35 | 88.62 | 10.24 | 65.40 | 80.42 |
| 74:08:34 | 99.63 | 84.25 | 5.29 | 70.90 | 84.00 |
| 84:44:04 | 99.41 | 83.76 | −7.85 | 72.51 | 82.56 |

### Results and Discussion

Several of the investigated microbes were capable of reducing butyric acid levels generated by lipase action in the laundry environment when applied as spores. Strains NRRL B50256 achieved a 99.74% reduction of butyric acid after 31.77 hours and continued to reduce 58.23% after 84.73 hours. NRRL B-67161 did not demonstrate consistent reduction of butyric acid indicating that this property is not characteristic of all microbes. NRRL B-67163 demonstrated a 79.37% reduction of butyric acid after 84.73 hours. NRRL B-67164 achieved an 85.06% reaction of butyric acid after 84.73 hours (Table 7).

With respect to caproic acid reduction, NRRL B50256 achieved 95.66% reduction by 42.4 hours and continued to suppress 83.76% after 84.7 hours. NRRL B-67161 did not demonstrate consistent reduction of caproic acid indicating that this property is not characteristic of all microbes. NRRL B-67163 achieved a 72.51% reaction after 84.73 hours. NRRL B-67164 achieved an 84.00% reduction by 74.1 hours and maintained an 82.56% reduction at 84.7 hours (Table 9).

### DEPOSIT OF BIOLOGICAL MATERIAL

The following biological materials were deposited under the terms of the Budapest Treaty at the Agricultural Research Service Culture Collection (NRRL) of the U.S. Department of Agriculture located at 1815 North University Street, Peoria, Ill. 61604 U.S.A., and given the following accession numbers:

| Deposit | Accession Number | Date of Deposit |
|---|---|---|
| Pseudomonas monteilii | NRRL B-50256 | Feb. 18, 2009 |
| Lysinibacillus contaminans | NRRL-B-67160 | Dec. 10, 2015 |
| Bacillus subtilis subsp. subtilis | NRRL-B-67161 | Dec. 10, 2015 |
| Bacillus subtilis | NRRL-B-67162 | Dec. 10, 2015 |
| Bacillus subtilis | NRRL-B-67163 | Dec. 10, 2015 |
| Bacillus subtilis | NRRL-B-67164 | Dec. 10, 2015 |

The strain has been deposited under conditions that assure that access to the culture will be available during the pendency of this patent application to one determined by foreign patent laws to be entitled thereto. The deposit represents a substantially pure culture of the deposited strain. The deposit is available as required by foreign patent laws in countries wherein counter parts of the subject application or its progeny are filed. However, it should be understood that the availability of a deposit does not constitute a license to practice the subject invention in derogation of patent rights granted by governmental action.

Lysinibacillus sp. 62733 is another name for the strain Lysinibacillus contaminans deposited under accession number NRRL-B-67160.

1-4. (canceled)

5. A composition that is a detergent composition or a rinse aid composition, comprising one or more isolated cultures selected from the group consisting of:
a strain having the deposit accession number NRRL-B-67164;
a strain having the deposit accession number NRRL B-50256;
a strain having the deposit accession number NRRL-B-67163;
a strain having the deposit accession number NRRL-B-67162;
a strain having the deposit accession number NRRL-B-67160;
a strain having the deposit accession number NRRL-B-67161;
a mutant of at least one of the deposited strains, wherein the mutant has all of the identifying characteristics of the corresponding deposited strain;
a progeny of one of the deposited strains; and
a mixture of two or more of the strains, mutants, or progeny; and
if the composition is a detergent composition, the composition additionally comprising at least one detergent component; and
if the composition is a rinse aid composition, the composition additionally comprising a fabric softener or fabric conditioner.

6-7. (canceled)

8. The composition of claim 5, wherein the isolated culture is present as a vegetative, a dormant or a spore culture.

9. The composition of claim 5, wherein the detergent component is selected from the group consisting of surfactants, builders, flocculating aid, chelating agents, dye transfer inhibitors, enzymes, enzyme stabilizers, enzyme inhibitors, catalytic materials, bleach activators, hydrogen peroxide, sources of hydrogen peroxide, preformed peracids, polymeric dispersing agents, clay soil removal/anti-redeposition agents, brighteners, suds suppressors, dyes, perfumes, structure elasticizing agents, fabric softeners, carriers, hydrotropes, builders and co-builders, fabric huing agents, anti-foaming agents, dispersants, processing aids, and pigments.

10. The composition of claim 5, wherein the composition further comprises one or more enzymes selected from the group consisting of amylases, arabinases, carbohydrases, cellulases, cutinases, DNases, galactanases, lipases, mannanases, oxidases, pectinases, proteases, and xylanases.

11. The composition of claim 10, wherein the composition comprises a lipase.

12. (canceled)

13. The composition of claim 5, wherein the concentration of each of the isolated cultures in the composition is in the range of 1×10⁴ to 1×10¹² cells per 100 mL.

14-26. (canceled)

27. A method for washing an item, comprising:
a. exposing the item to a wash liquor, wherein the wash liquor includes the detergent composition of claim 5; and
b. completing at least one wash cycle.

28. The method of claim 27, wherein the item is a textile, a fabric, a dishware or a hard surface.

29. The method of claim 28, wherein the hard surface is the interior of a laundry washing machine or an automated dish washing machine.

30. (canceled)

31. The method of claim 27, wherein the concentration of each of the cultures in the wash liquor is in the range of 1×10⁴ to 1×10¹² cells per 100 mL.

32. The method of claim 31, wherein the concentration of the cultures in the wash liquor is in the range of 1×10⁴ to 1×10⁶ cells per 100 mL.

33. The method of claim 27, wherein the method reduces the amount of free fatty acids from items comprising free fatty acids.

34. The method of claim 33, wherein the method reduces the amount of free fatty acids having from 2 to 10 carbon atoms.

35. The method of claim 34, wherein the free fatty acids are selected from the group consisting of formic acid, acetic acid, butyric acid, valeric acid, caproic acid, enanthic acid, caprylic acid, pelargonic acid and capric acid.

36. The method of claim 35, wherein the amount of butyric acid or caproic acid is reduced at least 50% when measured with AssayII.

37. The method of claim 27, including, after step (b):
c. rinsing the item with water and the rinse aid composition of claim 5.

38. A method for rinsing an item, comprising:
for an item that is subjected to a wash cycle, rinsing the item with the rinse aid of claim 5.
